(19) United States
(12) Patent Application Publication
(10) Pub. No.: US 2024/0309422 A1
(43) Pub. Date: Sep. 19, 2024

(54) METHOD FOR DETECTING A PHAGE OR ANTIBIOTIC SENSITIVITY WITH RESPECT TO A BACTERIAL STRAIN

(71) Applicants: COMMISSARIAT A L'ENERGIE ATOMIQUE ET AUX ENERGIES ALTERNATIVES, Paris (FR); UNIVERSITE GRENOBLE ALPES, Saint Martin D'heres (FR); CENTRE NATIONAL DE LA RECHERCHE SCIENTIFIQUE, Paris (FR); INSTITUT POLYTECHNIQUE DE GRENOBLE, Grenoble (FR)

(72) Inventors: Pierre MARCOUX, Grenoble cedex (FR); Yoann ROUPIOZ, Theys (FR); Marc ZELSMANN, Biviers (FR); Emmanuel PICARD, Grenoble cedex (FR); Larry O’CONNELL, Londres (GB)

(73) Assignees: COMMISSARIAT A L'ENERGIE ATOMIQUE ET AUX ENERGIES ALTERNATIVES, Paris (FR); UNIVERSITE GRENOBLE ALPES, Saint Martin D'heres (FR); CENTRE NATIONAL DE LA RECHERCHE SCIENTIFIQUE, Paris (FR); INSTITUT POLYTECHNIQUE DE GRENOBLE, Grenoble (FR)

(21) Appl. No.: 18/604,656

(22) Filed: Mar. 14, 2024

(30) Foreign Application Priority Data
Mar. 14, 2023 (FR) 23 02364

(51) Int. Cl. C12Q 1/18; C12M 1/00; C12M 1/32; C12Q 1/70

(52) U.S. Cl. CPC C12Q 1/18; C12M 23/12; C12M 23/22; C12M 23/34; C12M 41/06; C12Q 1/70

(57) ABSTRACT

A method for detecting a lytic activity of one or more types of phages or of the activity of one or more antibiotics with respect to a bacterial strain, the method including using a first multiwell plate, each well receiving a sample containing phages or antibiotics, making at least one deposit on a transparent area of a support, the deposit containing bacteria of the bacterial strain, applying the first plate against the support so that each sample in each well comes into contact with the deposit, for each well, determining, using the detection means, the activity or absence of activity of the phage or antibiotic with respect to the bacterial strain, taking into account the intensity of light signals transmitted through each well and/or the variation thereof over time.

### TECHNICAL FIELD OF THE INVENTION

The present invention relates to a method for detecting the sensitivity of phages or antibiotics with respect to a bacterial strain.

### PRIOR ART

Phage therapy is a medical approach used to treat infectious diseases of bacterial origin, relying on the natural abilities of certain viruses, called bacteriophages (and commonly called phages), to destroy the bacteria that they specifically recognize.

The implementation of this approach requires the selection beforehand of the phages that have a particular sensitivity for the bacterium to be destroyed. A conventional method consists in using a well plate, in which each well is filled with a suspension of bacteria. Phages that are distinct in terms of composition and/or concentration are then added to each well. Each well is then monitored to determine whether or not a lysis (destruction) reaction occurs. When lysis occurs in a well, it can be deduced therefrom that the phage added at the defined concentration has affinity and lytic activity for the bacterium. It will therefore potentially be usable for the implementation of phage therapy. Patent application EP3822360A1 describes a solution using a principle of detecting lytic activity of phages by lensless imaging. A particular embodiment consists in placing a fluidic chip having several chambers between a light source and an image sensor. In an exemplary embodiment, bacteria of one and the same bacterial strain are placed in each chamber and phages of distinct viral strains, or of one and the same viral strain are added thereto, but at distinct concentrations. The reaction which occurs in each chamber is then observed. Using the image sensor, it is determined whether the bacterium is:
barely or not at all sensitive to the viral strain of the phage when the detected light intensity decreases between two successive measurement times, or
sensitive to the viral strain of the phage when the detected light intensity does not decrease or increases between two successive measurement times.

This prior solution may present risks of pollution of the samples, each chamber possibly being exposed to the external environment.

Patent application WO2011/009213 relates to a method for testing affinities between biofilms and antimicrobial agents.

The aim of the invention is to propose a solution which makes it possible to detect an interaction between a bacterial strain and bacteriophages or antibiotics that are distinct in terms of composition/concentration, said solution being:
automated or easy to automate;
simple and reliable to implement, making it possible to parallelize distinct detection areas, limiting the risks of cross-contamination, while not compromising bacterial growth;
fast to implement and making it possible to test a large number of combinations, while limiting the number of manipulations to be performed.

The ultimate aim of the invention is to identify, in a parallel manner, the types of phages possessing lytic activity toward a targeted bacterium. The results thus produced then make it possible to define the combinations of phage types (“phage cocktails”) capable of having a therapeutic activity in vivo.

The invention thus makes it possible to determine the impact of various concentrations of a panel of antibacterial agents (antibiotics or bacteriophages), on the biomass of a bacterial strain.

### SUMMARY OF THE INVENTION

This aim is achieved by means of a method for detecting a lytic activity of one or more types of phages or of the activity of one or more antibiotics with respect to a bacterial strain, the phages or antibiotics optionally being distinct from each other in terms of composition and/or concentration, the method consisting in:
using a first multiwell plate, each well being made of a material transparent to the light signals emitted by a light source and permeable to apolar gases, each well receiving a sample containing phages or antibiotics,
making at least one deposit on a transparent area of a support, the deposit containing bacteria of the bacterial strain,
applying the first plate against the support so that each sample in each well comes into contact with said deposit,
placing the assembly formed by the first plate and the support between a light source and light detection means,
emitting light signals through each well using the light source,
for each well, determining, using the detection means, the activity or absence of activity of the phage or antibiotic with respect to said bacterial strain, taking into account the intensity of the transmitted light signals and/or the variation thereof over time.

According to one particularity, the phages or antibiotics are present in each well in the form of a hydrogel.

According to another particularity, the bacteria are dispersed in a gel deposited on said second plate so as to form said deposit.

According to another particularity, the support is made in the form of a second plate compartmentalized by means of leaktight partitions so as to form several distinct compartments, said deposit being subdivided into several distinct deposits added to each compartment of the second plate.

According to another particularity, the first plate is held against said second plate so that the phages or antibiotics contained in each well come into contact with a deposit placed in a compartment separate from the second plate.

According to another particularity, the first plate is turned upside down and moved so as to come to bear, via an open face on said wells, against a functional face of the support carrying the deposit.

According to another particularity, the support is turned upside down so as to come to bear, via a functional face carrying said deposit, against a face of the first plate open on said wells.

According to another particularity, the method comprises a step of relative displacement of the assembly formed by the first plate and the support relative to the detection means for the purpose of transmitting the light signals through each well of the first plate. According to another particularity, the detection means comprise an image sensor. According to another particularity, the image sensor is configured to acquire a first image at a first time and a second image at a later second time and in that a processing unit is configured to determine that the bacterial strain is considered to be:
barely or not at all sensitive to the phages or antibiotics when the intensity of the detected light signals decreases between said two times, or
sensitive to the phages or antibiotics when the intensity of the detected light signals does not decrease or increases between said two times.

According to another particularity, the image sensor cooperates with the light source according to a lensless imaging principle.

According to another particularity, the light source is configured to emit in a spectral band between 500 nm and 600 nm.

### BRIEF DESCRIPTION OF THE FIGURES

Other characteristics and advantages will become apparent from the following detailed description, which is given with reference to the appended figures listed below:

FIG. 1 schematically shows the detection system of the invention;

FIG. 2 illustrates the implementation of the detection method of the invention.

### DETAILED DESCRIPTION OF AT LEAST ONE EMBODIMENT

The invention relates to a method which is suitable for:
detecting the sensitivity, with respect to bacteria of one and the same bacterial strain, of phages derived from distinct viral strains;
detecting the sensitivity, with respect to bacteria of one and the same bacterial strain, of phages derived from one and the same viral strain at different concentrations;
detecting the sensitivity, with respect to bacteria of one and the same bacterial strain, of phages derived from distinct viral strains and at different concentrations.

The same principle can be applied for antibiotics with respect to bacteria of one and the same bacterial strain. In the remainder of the description, phages will be mentioned in a general manner, in order to cite the principles of detection of the method, defined above, but it should be understood that the invention can be applied identically to antibiotics.

The method of the invention makes it possible, on one and the same device, to bring bacteria B into contact with suspensions of phages P, which are distinct in terms of composition and/or concentration, in a simple manner, while forming distinct detection areas.

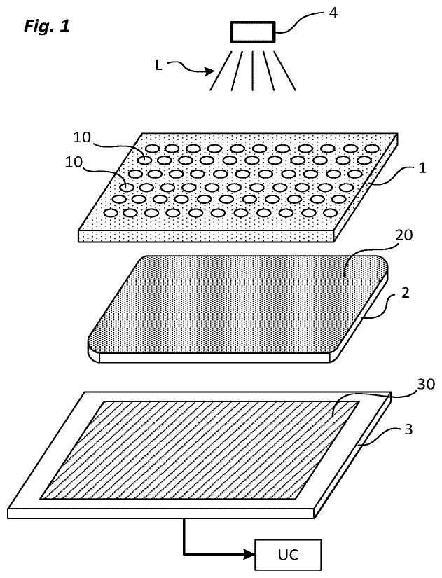

The method is implemented using the detection system that can be seen in FIG. 1. This system comprises a first well plate 1. This first plate 1 thus comprises several juxtaposed wells 10. The term “well” is understood to mean that the plate 1 comprises several distinct cavities, physically separated or sufficiently spaced out from one another. At each of its wells, this plate 1 has the particularity of being made of a material:
which is transparent, preferentially in the visible and/or near infrared range, so as to let light signals pass through;
permeable to apolar gases, notably to dioxygen and carbon dioxide in order to guarantee bacterial growth; it will be seen that this characteristic is important because it makes it possible to guarantee bacterial growth even during the implementation of the method of the invention.

In a non-limiting manner, the first plate 1 is, for example, made entirely of a material such as PDMS (polydimethylsiloxane) which has the characteristics defined above. This material also has the advantage of being biocompatible.

According to the method of the invention, each well 10 is filled with a suspension of phages. From one well to another, the suspensions of phages P may be distinct in terms of composition and/or concentration.

It should be noted that the plate 1 may be in the form of a device prefilled with the phages P and thus ready for use. The plate 1 is, for example, kept sealed pending its use. The phages P can be dispersed in a hydrogel added in an appropriate amount to each well 10 of the plate 1. This hydrogel may be a matrix made of agar-agar. Since this matrix is composed of about 99% water, the phages P are stored in a saline aqueous environment, which helps to maintain the infectious titre of the phages over a long period of time.

The plate 1 may comprise several tens of juxtaposed wells 10. This number may notably depend on the size of the sensor 3 used for detection.

As indicated above, a material such as PDMS is impermeable to water but permeable to apolar gases, thereby allowing gas exchanges at the wells 10 and thus ensuring bacterial growth, even when the phages P and the bacteria B are brought into contact. In the context of the invention, each well 10 thus materializes a delimited and distinct detection area through which the light source 4 can transmit light signals.

The system comprises a support for one or more deposits containing bacteria B of the bacterial strain. In a non-limiting manner and in the remainder of the description, this support may be formed of a second plate 2 capable of receiving, over its entire surface or its functional volume, at least one deposit 20 of material. The support could also be formed directly by the surface of the sensor (see below).

The deposit 20 advantageously has a thickness and a viscosity that are sufficient to form a lawn. This deposit 20 may be in the form of a gel containing the bacteria. It can be a gelatinous substance such as agar-agar, which is commonly used to grow bacteria. In a known manner, this substance is produced from a red alga (compatible with the level of transparency required for detection) which provides a suitable growth surface for many varieties of bacteria. Of course, it would be possible to use another substance.

It should be noted that it is possible to produce a single deposit 20 which covers the entire functional surface of the second plate 2. It is also possible to produce several juxtaposed deposits, each deposit being intended to be associated with a distinct well of the first plate. To accommodate each of these deposits, the second plate 2 may have compartments, the compartments being partitioned in a leaktight manner with respect to one another. This compartmentalized version advantageously makes it possible to prevent any diffusion during the implementation of the method of the invention. It should in fact be noted that it is necessary to prevent the reaction localized in a well from spreading over an adjacent well, that is to say that phages contained in a well do not spread beyond their well, contaminating an adjacent well.

Each deposit 20 contains a non-zero concentration of bacteria B.

The second plate 2, and also the deposit 20, are selected to be transparent, preferentially in the visible and/or near infrared range, to the light signals L of the light source (see below) so that these light signals L can at least partially pass through them during the implementation of the method. In a non-limiting manner, the second plate may also be made of PDMS. However, other materials, such as PMMA (polymethyl methacrylate) or COC (cyclic olefin copolymer) could be envisaged.

The detection system comprises a light source 4. The light source 4 is for example composed of one or more light-emitting diodes or laser diodes. A bandpass filter (not shown-possibly integrated into the source) may be used at the output of the light source 4 or at the input of the image sensor 3, to adjust the spectral band of the emitted light signals. Preferably, the light signals L emitted by the light source 4 extend along an illumination spectral band of the visible and near infrared range. It is preferably between 400 nm and 1000 nm, preferably between 500 nm and 600 nm.

The detection system thus comprises an image sensor 3. This image sensor 3 may be a CCD or CMOS type sensor. In a non-limiting manner, it has, for example, a resolution of 5344×3516 pixels. It should be noted that the system advantageously does not include magnification optics, the latter operating on the principle of lensless imaging.

The image sensor 3 is connected to a processing unit UC, which receives the images acquired by the image sensor. The image sensor 3 is capable of generating several images at successive times, thus making it possible to monitor the progression of the reactions in each well 10.

It would be possible to use detection means other than the image sensor 3, for example a conventional microscope. The lensless imaging detection solution has the advantage of being able to monitor a large number of wells simultaneously, possibly without installing complex mechanical means if all the elements of the system are correctly proportioned. It also allows simple image processing.

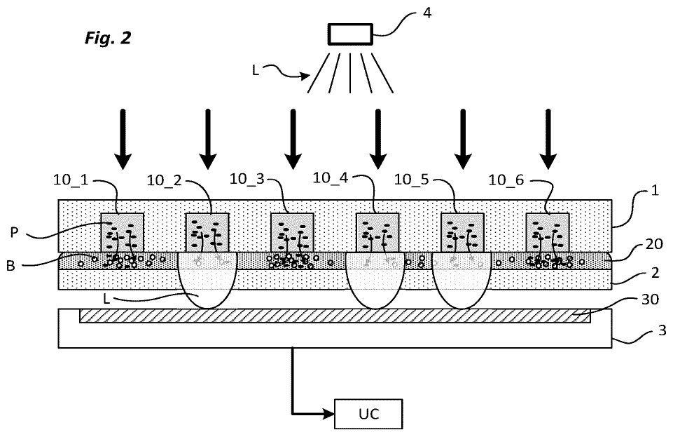

Starting from the various elements of the system as described above, the detection method is implemented in the manner described below, with reference to FIG. 2. The first plate 1 is, for example, turned upside down and brought to bear, via its open face on the wells, against the functional face of the second plate 2 carrying the deposit 20, in such a way that the container of each well 10 comes into contact with the deposit 20 present on the functional surface of the second plate 2. The phages P present in each well 10 are thus brought into contact with the bacteria B inoculated in the deposit 20 of the second plate. The two plates 1, 2 are, for example, held against each other so as to create a seal from one well to another and to make it possible to obtain sufficient diffusion of the phages P into the inoculated deposit of bacteria B.

The permeability to apolar gases of the first plate 1 makes it possible to ensure bacterial growth, even when the phages P and the bacteria B are brought into contact, by applying the first plate 1 carrying the phages against the support of the bacteria B. The assembly, for example formed by the two plates, is positioned between the light source 4 and the image sensor 3. It is for example positioned as close as possible to the surface 30 of the sensor 3, materialized for example by a protective window, for example on this surface or just above it. As already indicated above, it should be noted that the deposit 20 could be placed directly on the surface 30 of the sensor 3, the latter then serving as a support for the bacteria B.

The light source 4 is activated so as to illuminate the various wells 10. The illumination can make it possible to illuminate all the wells 10 simultaneously or one after the other or in groups of several wells. In the latter case, actuating means may be provided to perform a movement of the assembly formed by the two plates and/or of the light source 4.

The image sensor 3 is activated so as to capture, simultaneously or quasi-simultaneously, images of each well 10.

In the absence of phages, or in the absence of notable effects of the phages P, on the bacterial strain, the bacteria proliferate. When phages P exhibit a particular sensitivity for the bacterial strain and a lytic activity on the latter, the number of bacteria B decreases. The increase or decrease in the number of bacteria B in each well 10 then leads to a variation in the nature and/or intensity of the light transmitted through each well 10. The images acquired by the sensor 3, over time, thus allow this evolution to be assessed and quantified. In other words, at each well 10, if the phage P exhibits a particular lytic activity with respect to the bacterial strain, the latter will tend to lyse the bacteria B. The intensity of the light emitted by the light source 4 through the well 10 and captured by the image sensor will increase.

Conversely, if the phage P present in said well exhibits only a low lytic activity, or even no lytic activity, for the bacterial strain, the intensity of the light emitted by the light source 4 through the well 10 and captured by the image sensor 3 will be stable or will decrease, because of the proliferation of the bacteria B. The detection principles are described, for example, in patent application EP3822360A1.

In FIG. 2, it can thus be seen that, at each well 10, the phages diffuse into the deposit 20 inoculated with bacteria B. Some phages P which exhibit sensitivity with respect to the bacterial strain destroy the bacteria B and others which do not have said sensitivity will cohabit with bacteria B, the bacteria B then being able to continue their proliferation. In this FIG. 2, the phages P initially present in wells 10_2, 10_4, 10_5 exhibit sensitivity for the bacterial strain and lytic activity on the bacteria. The light signals transmitted through these wells are of a higher intensity than those transmitted through the other wells 10_1, 10_3, 10_6, for which the phages P (in terms of composition and/or in terms of concentration) do not exhibit a particular sensitivity with respect to the bacterial strain. At the wells 10_1, 10_3, 10_6, the light signals are in fact attenuated by the compounds (bacteria+phages) present.

The same principle applies with a second compartmentalized plate.

To image all the wells, it should be noted that it is also possible to move the image sensor 3 and/or the first plate/second plate assembly, if it proves to be the case that the capture surface 30 is not sufficient to display all the wells 10 in a single image.

The invention makes it possible to rapidly test the lytic activity of phages P with respect to a bacterial strain. The test can be performed in just a few hours.

By way of example, using an 864 mm² image sensor, it was possible to monitor sixty-six wells simultaneously. Of course, it would be possible to increase the number of wells on one and the same plate, and/or to increase the surface area of the sensor and/or even to use several juxtaposed sensors.

It should also be noted that the principle of the invention makes it possible to monitor in real time the lytic activity of the phages on the bacteria. The kinetic information from the real-time acquisition thus provides an additional layer of information.

The invention has many advantages, among which are:
the multiplexing of the measurements of phage activity on the bacterial strains,
the use of conventional components such as a well plate, an image sensor, a light source,
a method that is easy to implement and capable of delivering results quickly,
a reliable method since it requires only few manipulations,
a method allowing continuous monitoring, which can give faster results when implemented in an incubator.

1. A method for detecting a lytic activity of one or more types of phages or of the activity of one or more antibiotics with respect to a bacterial strain, the phages or antibiotics optionally being distinct from each other in terms of composition and/or concentration, wherein the method comprises:
using a first multiwell plate, each well being made of a material transparent to the light signals emitted by a light source and permeable to apolar gases, each well receiving a sample containing phages or antibiotics,
making at least one deposit on a transparent area of a support, the deposit containing bacteria of the bacterial strain,
applying the first plate against the support so that each sample in each well comes into contact with said deposit,
placing the assembly formed by the first plate and the support between a light source and light detection means,
emitting light signals through each well using the light source,
for each well, determining, using the detection means, the activity or absence of activity of the phage or antibiotic with respect to said bacterial strain, taking into account the intensity of the transmitted light signals and/or the variation thereof over time.

2. The method according to claim 1, wherein the phages or antibiotics are present in each well in the form of a hydrogel.

3. The method according to claim 1, wherein the bacteria are dispersed in a gel deposited on said second plate so as to form said deposit.

4. The method according to claim 1, wherein the support is made in the form of a second plate compartmentalized with leaktight partitions so as to form several distinct compartments, said deposit being subdivided into several distinct deposits added to each compartment of the second plate.

5. The method according to claim 4, wherein the first plate is held against said second plate so that the phages or antibiotics contained in each well come into contact with a deposit placed in a compartment separate from the second plate.

6. The method according to claim 1, wherein the first plate is turned upside down and moved so as to come to bear, via an open face on said wells, against a functional face of the support carrying the deposit.

7. The method according to claim 1, wherein the support is turned upside down so as to come to bear, via a functional face carrying said deposit, against a face of the first plate open on said wells.

8. The method according to claim 1, it wherein the method comprises a step of relative displacement of the assembly formed by the first plate and the support relative to the detection means for the purpose of transmitting the light signals through each well of the first plate.

9. The method according to claim 1, wherein the detection means comprise an image sensor.

10. The method according to claim 9, wherein the image sensor is configured to acquire a first image at a first time and a second image at a later second time and in that a processing unit is configured to determine that the bacterial strain is considered to be:
barely or not at all sensitive to the phages or antibiotics when the intensity of the detected light signals decreases between said two times, or
sensitive to the phages or antibiotics when the intensity of the detected light signals does not decrease or increases between said two times.

11. The method according to claim 9, wherein the image sensor cooperates with the light source according to a lensless imaging principle.

12. The method according to claim 1, wherein the light source is configured to emit in a spectral band between 500 nm and 600 nm.
